(19) United States
(12) Patent Application Publication
(10) Pub. No.: US 2016/0113812 A1
(43) Pub. Date: Apr. 28, 2016

(54) Therapeutic Suit

(71) Applicant: Clementine L. Bender, Lago Vista, TX (US)

(72) Inventor: Clementine L. Bender, Lago Vista, TX (US)

(21) Appl. No.: 14/582,690

(22) Filed: Dec. 24, 2014

Related U.S. Application Data
(60) Provisional application No. 62/069,411, filed on Oct. 28, 2014.

(51) Int. Cl. A61F 7/08; A61F 7/10

(52) U.S. Cl. CPC A61F 7/08; A61F 7/10; A61F 2007/108; A41D 2400/32

(57) ABSTRACT

The therapeutic suit reliefs joint pain by applying heat or cold relief to one or all joints of the body and bringing healing to joints by vasoconstriction, centralizing oxygenated blood around the joints during cold therapy treatment.

### RELATED APPLICATIONS

There are no current pending applications.

### I. FIELD OF THE INVENTION

The present invention relates generally to a therapeutic suit having integral overlapping pockets, each adaptable to receive thermal regulation packs.

### II. MOTIVATION FOR THE INVENTION

Individuals who have experienced pain, discomfort, or debilitating injuries of the joints commonly worry about flare-ups and constantly search for devices, medicine, or other forms of relief. Such individuals would find the present invention useful. Sufferers of diseases or inflictions such as rheumatoid arthritis, fibromyalgia, or other chronic illnesses, and even sports- or work-related injuries or accidents can benefit from this invention. Locating and pin-pointing relief at specific areas of the body with a single, fits-most-style of clothing would be extremely beneficial to sufferers of ailments described above. The ability to place and remove either hot or cold packs directly on the problem site by inserting into pockets is time saving and an effective way of treating pain.

### III. DESCRIPTION OF THE DRAWINGS

The advantages and features of the present invention will become better understood with reference to the following more detailed description and claims taken in conjunction with the accompanying drawings, in which like elements are identified with like symbols, and in which:

FIG. 1 is a front view of a therapeutic suit 10 in accordance with the preferred embodiment of the present invention;

FIG. 2 is a rear view of the therapeutic suit 10 in accordance with the preferred embodiment of the present invention; and,

FIG. 3 is an isolated view of a thumb aperture 54 of the therapeutic suit 10 in accordance with the preferred embodiment of the present invention.

DESCRIPTIVE KEY

| | |
|---|---|
| 10 | therapeutic suit |
| 20 | top |
| 22 | front |
| 24 | rear |
| 26 | body |
| 28 | waist |
| 32 | neck |
| 34 | wrist |
| 36 | zipper |
| 38 | seam |
| 38b | sleeve seam |
| 38c | inseam |
| 38d | outer pant seam |
| 38e | front center seam |
| 38f | rear center seam |
| 42 | first sleeve |
| 44 | second sleeve |
| 46 | thumb aperture |
| 48 | hem |
| 52 | first wrist pocket |
| 54 | second wrist pocket |
| 56 | first elbow pocket |
| 58 | second elbow pocket |
| 62 | first front shoulder pocket |
| 64 | second front shoulder pocket |
| 66 | first rear shoulder pocket |
| 68 | second rear shoulder pocket |
| 72 | spine pocket |
| 74 | pocket face |
| 76 | pocket cover |
| 82 | first pocket fastener |
| 84 | second pocket fastener |
| 90 | pants |
| 92 | ankle |
| 94 | first leg |
| 96 | second leg |
| 102 | first front ankle pocket |
| 104 | second front ankle pocket |
| 106 | first rear ankle pocket |
| 108 | second rear ankle pocket |
| 112 | first knee pocket |
| 114 | second knee pocket |
| 116 | first hip pocket |
| 118 | second hip pocket |
| 120 | gel pack |
| 150 | wearer |
| 155 | thumb |

### IV. DESCRIPTION OF THE INVENTION

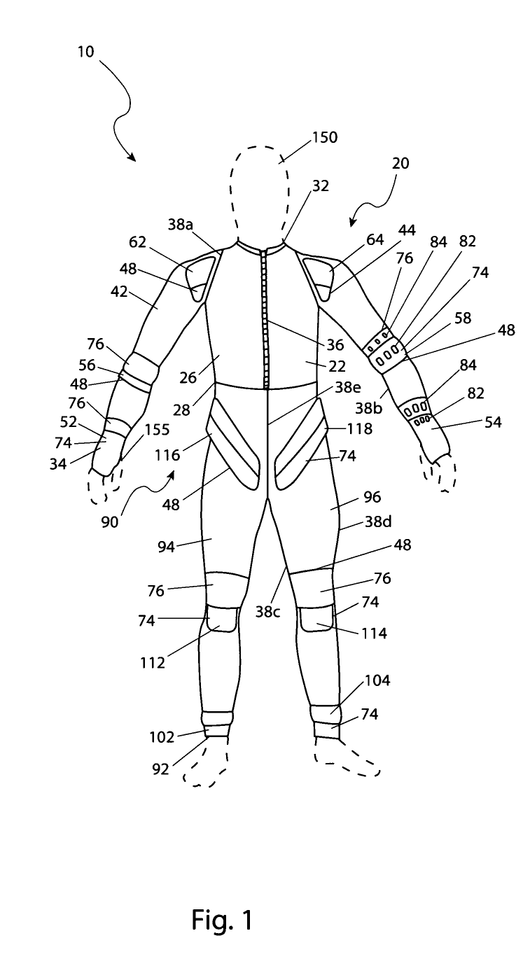

The best mode for carrying out the invention is presented in terms of its preferred embodiment, herein depicted within FIGS. 1 through 3. However, the invention is not limited to the described embodiment, and a person skilled in the art will appreciate that many other embodiments of the invention are possible without deviating from the basic concept of the invention and that any such work around will also fall under scope of this invention. It is envisioned that other styles and configurations of the present invention can be easily incorporated into the teachings of the present invention, and only one particular configuration shall be shown and described for purposes of clarity and disclosure and not by way of limitation of scope.

The terms “a” and “an” herein do not denote a limitation of quantity, but rather denote the presence of at least one of the referenced items.

### 1. Detailed Description of the Figures

The present invention describes a therapeutic suit (herein referred to as the “device”) 10, which provides a means to treat joint pain caused by overuse or osteoarthritis in multiple joints throughout the body simultaneously by positioning gel packs 120 into various pockets 52, 54, 56, 58, 62,64, 66, 68, 72, 102, 104, 106, 108, 110, 112, 114, 116, 118 distributed around the top 20 and pants 80. The gel packs 120 may be heated or chilled by some external device at the selection of a user. It should be noted that since the size of the users to which this device 10 may be useful can vary considerably there must be a range of sizes implicit in the preferred embodiment without limiting the scope of the invention. Such size variability may include accommodations for girth as well as for stature.

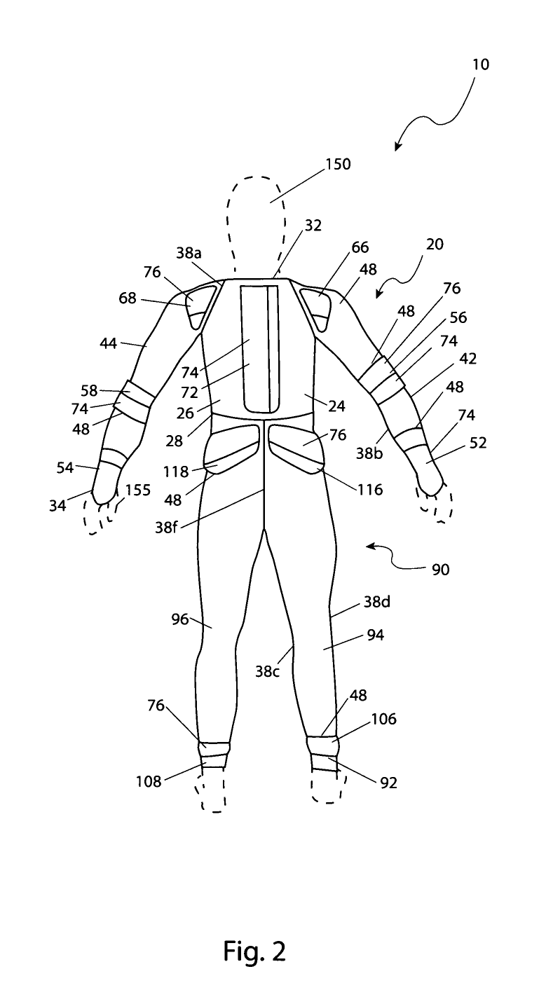

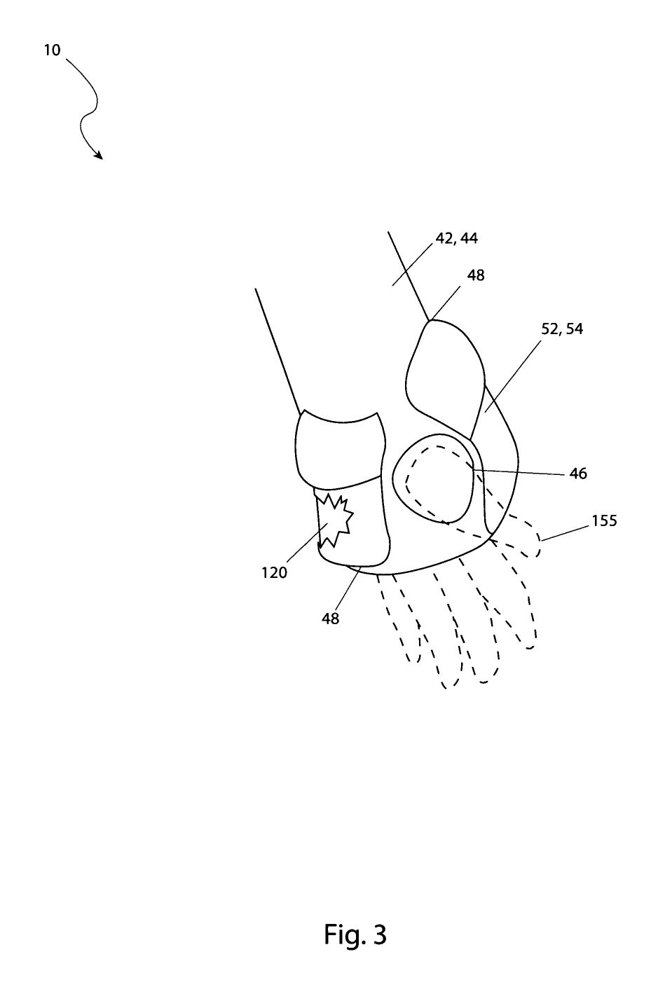

Referring now to FIG. 1, a front view, and FIG. 2, a rear view of the device 10, according to the preferred embodiment of the present invention, are disclosed. The device 10 includes a top 20 and pants 80 composed preferably of SPANDEX®, or other similar material, so as to result in a close fitting garment. The top 20 is configured to have a wrap-around body 26 with a zipper 36 extending from a waist 28 to a neck 32. The top 20 is provided with a first sleeve 42 and a second sleeve 44 so as to fit the upper torso and arms of a wearer 150. In a preferred embodiment, the sleeves 42, 44 are full-length with a raglan-type seam 38a at a juncture with the body 26. The seams 38 are configured to be a connection, preferably by stitching, along an edge of a piece, or pieces, of the constituent material comprising the device 10 in order to fasten the piece, or pieces together. The sleeves 42, 44 are each composed of a single piece of SPANDEX® with a sleeve seam 38b on a lower side of the sleeve 42, 44 extending from the wrist 34 to the body 26. Disposed at a distal end of each sleeve 42, 44 is a thumb aperture 46 configured for the insertion of the thumb 155 of the wearer as seen in FIG. 3. The thumb 155 inserted into the thumb aperture 46 maintains the length of the sleeve 42, 44 and assures the correct positioning of the wrist pockets 52, 54 located at the distal end of each respective sleeve 42, 44 as well as the elbow pockets 56, 58.

A first wrist pocket 52 is disposed at the distal end of the first sleeve 42. A second wrist pocket 54 is similarly disposed at the distal end of the second sleeve 44. The wrist pockets 52, 54 are each configured to be a pocket face 74 attached to the respective sleeve 42, 44, preferably by stitching, around three (3) sides. The wrist pockets 52, 54 are provided with hems 48 along both lateral edges with the material of the respective sleeve 42, 44 as well as a hem 48 along the distal edge of each wrist pocket 52, 54 with the material at, and parallel to, the distal edge of each respective sleeve 42, 44. The lateral hems 48 are located in proximity to the thumb aperture 46 of each sleeve 42, 44. The pocket face 74 is also provided with an overlapping hem 48 with itself at a proximal edge of the wrist pocket 52, 54. The hems 48 are configured to be along an interior surface of the wrist pocket 52, 54. The width of the pocket face 74 of each wrist pocket 52, 54 is equivalent to the width of a gel pack 120 to be placed within the wrist pocket 52, 54.

An overlaying pocket cover 78 is attached to each respective sleeve 42, 44, preferably by stitching, in proximity to the pocket face 74 of each wrist pocket 52, 54. The pocket cover 76 is configured to overlap approximately fifty percent (50%) of the pocket face 74 having lateral hems 48 in conjunction with the hems 48 along the lateral edges of the pocket face 74 of each wrist pocket 52, 54 of each respective sleeve 42, 44. The pocket cover 76 is attached to each sleeve 42, 44, preferably by stitching, at a proximal side of the proximal edge of each respective wrist pocket 52, 54. The pocket cover 76 is intended to overlap a portion of the pocket face 74 and retain the enclosed gel pack 120 within each wrist pocket 52, 54.

Disposed along an inner overlapping face of the pocket cover 76, in proximity to the distal edge thereof, is a plurality of pocket first fasteners 82. The pocket first fastener 82 is configured to be a hooks portion of a hook-and-loop fastener such as VELCRO®. At a location on the pocket face 74 corresponding to the location of the pocket first fastener 82 on the pocket cover 76 is a pocket second fastener 84. The pocket second fastener 84 consists of the complimentary loops portion of a hook-and-loop fastener. The pocket first fastener 82 and the pocket second fastener 84 combine to secure a gel pack 120 within each wrist pocket 52, 54.

Disposed at a midpoint in the first sleeve 42, corresponding to the location of a wearer's elbow, is a first elbow pocket 56. The first elbow pocket 56 is constructed in a similar manner to the first wrist pocket 52. A pocket face 74 is attached, preferably by stitching, to the material of the first sleeve 42 at a lateral hem 48 along the seam 38a of the first sleeve 42. The pocket face 74 encircles the first sleeve 42 and is rejoined, again with a lateral hem 48 on an opposite side of the pocket face 74, to the seam 38a of the first sleeve 42. The pocket face 74 is stitched to the first sleeve 42 along a distal edge to form a hem 48 with the material of the first sleeve 42. A pocket cover 76 is attached near the proximal edge of the pocket face 74, preferably by stitching, to form a hem 48 with the material of the first sleeve 42. The pocket cover 76 is then stitched to the seam 38a of the first sleeve 42 along each lateral edge of the pocket cover 76. A plurality of pocket first fasteners 82 and pocket second fasteners 84 are stitched to the interior face of the pocket cover 76 and the exterior of the pocket face 74 respectively in order to secure a gel pack 120 within the first elbow pocket 56 of the first sleeve 42. The second elbow pocket 58, attached to the second sleeve 44 is constructed in a similar manner to the first elbow pocket 56 to retain another gel pack 120.

A first front shoulder pocket 62 and a second front shoulder pocket 64 are attached, preferably by stitching, at an upper portion of the front 22 of the top 20 in an area corresponding to the shoulder and clavicle of a wearer 150. A first rear shoulder pocket 66 and a second rear shoulder pocket 68 are disposed in a similar manner on an upper portion of the rear 24 of the top 20. The shoulder pockets 62, 64, 66, 68 are generally configured to conform to the pattern of the seams 38 of the top 20 resulting in an overall triangular shape. In an alternate embodiment the shoulder pockets 62, 64, 66, 68 may conform to a scalene trapezoidal shape. Each shoulder pocket 62, 64, 66, 68 is provided with a plurality of pocket first fasteners 82 (not shown) and pocket second fasteners 84 (not shown), similar to the first wrist pocket 52 in order to retain a gel pack 120 within the shoulder pockets 62, 64, 66, 68.

A spine pocket 72 is configured vertically along a central portion of the rear 24 of the top 20 extending from the waist 28 to the neck 32. The spine pocket 72 may be provided with additional stitching through the pocket face 74, perpendicular to a sagittal plane, so as to divide the spine pocket 72 into a plurality of smaller chambers more in keeping with a standard size of a gel pack 120.

It is envisioned that an alternate embodiment of the top 20 would be configured to have a foreshortened first sleeve 42 and second sleeve 44. The alternate sleeves 42, 44 would terminate at the upper arm of a wearer 150 and be provided with only shoulder pockets 62, 64, 66, 68 and a spine pocket 72.

The pants 90 are configured to be symmetrical about both a sagittal and a coronal plane having an inseam 38c along the inner part of each of the first leg 94 and the second leg 96 between the front 22 and the rear 24 as well as a front center seam 38e and a rear center seam 38f between the first leg 94 and the second leg 96 allowing sufficient flexibility to conform to the profile of a wearer 150. In a preferred embodiment, the pants 90 are configured to have a first leg 94 and a second leg 96 extending entirely to the ankle 92 of a wearer 150. The pants 90 may also be provided with some strap at the termination of the first leg 94, and the second leg 96 so as to be routed under each foot of a wearer 150 to secure the position of each leg 94, 96. Disposed at the distal end on a front 22 of the first leg 94 is a first front ankle pocket 102. The first front ankle pocket 102 is constructed similar to the first wrist pocket 52 having a pocket face 74 attached, preferably by stitching, to the material of the first leg 94 at a lateral hem 48 along the inseam 38c of the first leg 94. The pocket face 74 is attached to the distal end of the first leg 94 along a hem 48 at the ankle 92 and is joined with a lateral hem 48 on an opposite side of the first leg 94, to the outer leg seam 38d. A pocket cover 76 is attached near the proximal edge of the pocket face 74, preferably by stitching, to form a hem 48 with the material of the first leg 94. The pocket cover 76 is then stitched to the inseam 38 and to the outer leg seam 38d of the first leg 94 along each lateral edge of the pocket cover 76. A plurality of pocket first fasteners 82 and pocket second fasteners 84 are stitched to the interior face of the pocket cover 76 and the exterior of the pocket face 74 respectively in order to secure a gel pack 120 within the first front ankle pocket 102 of the first leg 94. The first rear ankle pocket 106 attached to the rear 24 of the first leg 94 is constructed in a similar manner to retain another gel pack 120. The second front ankle pocket 104, and a second rear ankle pocket 108 attached to the second leg 96 are constructed in a similar manner to retain a gel pack 120 in each pocket 104, 108.

Disposed at a midpoint on the front 22 of the first leg 94, corresponding to the location of a wearer's knee, is a first knee pocket 112. The first knee pocket 112 is constructed in a similar manner to the first front ankle pocket 102. The first knee pocket 112 may be slightly larger than the first front ankle pocket 102 so as to accommodate a larger gel pack 120. The second leg 96 is provided with a similarly constructed second knee pocket 114. The preferred embodiment does not include a chamber at the rear 24 of midpoint of either the first leg 94 or the second leg 96 for the insertion of a gel pack 120. However, this should not be seen as a limiting factor to the scope of the device 10 and it should be understood that any additional chambers may be included at any location in other embodiments of the device 10.

The first hip pocket 116 is located in proximity to the waist 28 of the pants 90 and is configured to extend from near the junction of the inseam 38d and the front center seam 38e of the first leg 94 at the front 22 of the pants 90 upwardly toward the waist 28 and outwardly to the outer leg seam 38d, crossing the outer seam 38 in proximity to the waist 28 and proceeding across the rear 24 of the pants 90 to the vicinity of the rear center seam 38f. The second hip pocket 118 is an opposite hand configuration on the second leg 96. The purpose of the hip pockets 116, 118 is to treat the area of the pelvis and lumbar region of the wearer 150. The hip pockets 116, 118 may be provided with additional stitching through the pocket face 73, perpendicular to a transverse plane, so as to divide the hip pocket 116, 118 into smaller chambers to more easily accommodate a gel pack 120.

It is envisioned that the device 10 may be worn in a reversed, or inside-out, configuration to make the device fit more closely to the wearer 150.

### 2. Operation of the Preferred Embodiment

In accordance with the invention, the illustrated embodiment can be utilized by an enabled individual in a simple and straightforward manner with little or no training. After initial purchase or acquisition of the device 10, it would be installed as indicated in FIGS. 1 and 2.

The method of installing and utilizing the device 10 may be achieved by performing the following steps: acquiring a model of the device 10 having a desired style to suit the taste of a user; installing the gel packs 120 as necessary into the first wrist pocket 52, the second wrist pocket 54, the first elbow pocket 56, the second elbow pocket 58, the first front shoulder pocket 62, the second front shoulder pocket 64, the first rear shoulder pocket 66, the second rear shoulder pocket 68, the spine pocket 72, the first front ankle pocket 102, the second front ankle pocket 104, the first rear ankle pocket 105, the second rear ankle pocket 108, the first knee pocket 112, the second knee pocket 114, the first hip pocket 116, and the second hip pocket 118; heating the device 10 by tumbling the device 10 in a heated clothes dryer, or alternately chilling the device 10 in a freezer compartment as necessary to comply with the desires of the wearer 150; donning the top 20 and the pants 90; and carrying on one's normal activities as the heated, or cooled gel packs 120 alleviate the joint pain.

For a preferred method of use, the pockets herein described, particularly the spine pocket 72, would best accommodate a gel pack 120 having a segmented or cellular configuration so as to prevent the gel pack 120 from sliding down to the bottom of the spine pocket 72 due to gravity.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The embodiments were chosen and described in order to best explain the principles of the invention and its practical application, to thereby enable others skilled in the art to best utilize the invention and various embodiments with various modifications as are suited to the particular use contemplated.

What is claimed is:

1. A therapeutic suit, comprising:
a top garment, having a pair of front panels, a rear panel, a first sleeve, a second sleeve, a thumb aperture at a distal end of each said first and second sleeve;
a plurality of top garment pockets, each located on an exterior of said top garment;
a bottom garment, comprising a first panel and a second panel, wherein said first panel and said second panel are conjoined to form a first leg portion and a second leg portion; and,
a plurality of bottom garment pockets, each located on an exterior of said bottom garment; wherein each of said plurality of top garment pockets and said bottom garment pockets are configured to receive a thermoregulating pack therein.

2. The therapeutic suit of claim 1, wherein said top garment is configured to have a wrap-around body with a fastener extending from a top waist area to a neck area.

3. The therapeutic suit of claim 2, wherein said plurality of top garment pockets further comprise:
a first wrist pocket disposed at said distal end of said first sleeve;
a second wrist pocket disposed at said distal end of said second sleeve
a first elbow pocket disposed along a midpoint of said first sleeve;
a second elbow pocket disposed along a midpoint of said second sleeve;
a first front shoulder pocket disposed at an upper portion of said front panel adjacent a proximal end of said first sleeve;
a second front shoulder pocket disposed at an upper portion of said front panel adjacent a proximal end of said second sleeve;
a first rear shoulder pocket disposed at an upper portion of said rear panel adjacent a proximal end of said first sleeve;
a second rear shoulder pocket disposed at an upper portion of said rear panel adjacent a proximal end of said second sleeve; and.
a spine pocket configured vertically along a central portion of said rear panel extending from said top waist area to said neck area.

4. The therapeutic suit of claim 2, wherein said plurality of bottom garment pockets further comprise:
a first front ankle pocket disposed on a front of said first panel located at a distal end of said first leg;
a second front ankle pocket disposed on a front of said second panel located at a distal end of said second leg;
a first rear ankle pocket disposed on a rear of said first panel located at a distal end of said first leg;
a second rear ankle pocket disposed on a rear of said second panel located at a distal end of said second leg;
a first knee pocket disposed on a front of said first panel located at a midpoint of said first leg;
a second knee pocket disposed on a front of said second panel located at a midpoint of said second leg;
a first hip pocket disposed on said first panel located adjacent to a lower waist area and extending from a front to a rear thereof and,
a second hip pocket disposed on said second panel located adjacent to a lower waist area and extending from a front to a rear thereof.
